(19) United States
(12) Patent Application Publication
(10) Pub. No.: US 2008/0206152 A1
(43) Pub. Date: Aug. 28, 2008

(54) In Vivo Expression Profiling

(75) Inventors: Ralf Hoffmann, Leverkusen (DE); Helga Hummel, Aachen (DE); Matthias Wendt, Wuerselen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/916,548

(22) PCT Filed: Jun. 1, 2006
(86) PCT No.: PCT/IB06/51750

§ 371 Date: Dec. 5, 2007

(30) Foreign Application Priority Data
Jun. 7, 2005 (EP) 05104917.9

(51) Int. Cl. A61B 5/055; A61K 49/00

(52) U.S. Cl. 424/9.341; 424/9.1; 424/9.351

(57) ABSTRACT

The present invention relates to in vivo expression profiling using a plurality of specific targeting moieties each labelled with a different compound which allows to identify simultaneously the binding of each targeting moiety to a target.

The invention relates methods and tools for improving diagnostic imaging in vivo. The invention further relates tools and methods for producing tools which allow accurate diagnostic imaging based on the simultaneous in vivo qualitative and/or quantitative detection of a plurality of biomedical targets or biomedical disease diagnosis targets such as the expression or non-expression of a plurality of genes or presence or absence of a plurality of gene products such as proteins, and carbohydrates or lipids or metabolites whether circulating or bound.

Diagnosis of a disorder such as cancer is based on a range of procedures including physical examination, biochemical and histopathological investigations, and diagnostic imaging techniques.

The final confirmation of a tumour disease requires the extraction of a tissue sample from the suspected body area by physical intervention (biopsy, surgery, etc). The characterization of the tissue by histology provides important parameters to classify the tumour according to the TNM classification system, which is still the golden standard for the definition of the appropriate therapeutic regime and the outcome prognosis of the disease. Histological analysis allows the classification of the tissue as malignant, benign or normal. Additionally, the degree of differentiation is determined, providing an indication of the aggressiveness of the investigated cancer cells. Developments over the last years make it possible to monitor metabolic changes of suspicious tissue thereby delivering important information of the investigated tissue status. All diagnostic information taken together allows a final staging of the investigated cancer, with a direct correlation of the determined stage to the required therapeutic approach, the clinical outcome and survival probability.

It has been shown recently that the classification of a tumour is possible by the investigation of the activity and the expression levels of a combination of several genes or proteins, so-called patterns of biomolecules or expression profiles [Van't Veer et al. (2002) Nature 415, 530-536; Van de Vijver et al (2002) New Engl. J. Med. 347, 1999-2009; Van't Veer (2003) Nature Med. 9, 999-1000]. The analyses of these patterns are done on patient serum or patient tissue of suspected affected body regions.

Current diagnostic imaging methods make use of targeted contrast agents which identify a target associated with a pathological condition. The detection of a single protein has limited use in discriminating between the hundreds of different cell types that exist and make up the organs of higher mammals. Nor can a single protein normally discriminate between different physiological states of a cell with an appropriately high sensitivity and/or specificity. For instance, all tumour markers used today for the diagnosis of cancerous masses (e.g., PSA for prostate cancer, CA-125 for ovarian tumours, etc.) suffer from low sensitivity and specificity to detect the disease correctly in the very early stages. Clinical outcome prognoses based on the presence of such proteins often show poor correlation to the individual disease progression.

In contrast, it has been shown recently that by using a combination of multiple proteins or equivalent analytes (DNA, RNA, metabolites) in vitro, the classification of e.g., tumour cells, based on molecular properties, into consistent sub-classes is possible. Several studies demonstrated in the past how to apply DNA arrays to the analysis and classification of cancer [Golub et al. (1999) Science 286, 531-537; Perou (1999) PNAS 96, 9212-9217; Alizadeh, et al. (2000) Nature 403, 503-511; Perou et al. (2000) Nature, 406, 747-752; Ross et al. (2000) Nature Genet. 24, 227-235; Bittner et al. (2000) Nature 406, 536-540; Unger et al. (2001) Breast Cancer Res., 3, 336-341; Ramaswamy (2001) PNAS, 98, 15149-15154; West et al. (2001) PNAS, 98, 11462-11467; Khan et al. (2001) Nature Med. 7, 673-679; Sorlie et al. (2001) PNAS, 98, 10869-10874; Perou et al. (2000) Trends Mol. Med. December 2000, 67-76; Alizadeh (2001) J. Pathol. 195, 41-52]. Although the single marker approach is valuable, it is based on a rationale that is an over-simplification of cancer aetiology and progression. It has been demonstrated that the outward manifestation of the cancer phenotype is the result of many interacting pathways and programs in both the cancer cell and the host. To capture a more complete picture of the molecular state of cancer, investigators in both the public and private sectors have turned to DNA arrays that can be used to survey patterns of expression for thousands of genes simultaneously.

In addition, it has been shown that also improved prognosis of the overall clinical outcome of a tumour disease is feasible based on the use of patterns comprising the status (e.g., expression level) of multiple molecules rather than using singular events [Shipp et al. (2002) Nature Med. 8, 68-74; Shipp et al. (2002) Nature, 415, 530-536; Pomeroy (2002) Nature, 415, 436-442; Beer et al. (2002) Nature Med. 8, 816-824; Rosenwald et al. (2002) New Engl. J. Med. 346, 1937-1947]. In the case of breast cancer for instance, the strongest predictors for metastases used clinically today (e.g. lymph node status, histological grade) fail to classify accurately breast tumours according to their clinical behaviour. By the use of in vitro gene expression analysis in primary breast tumours and the application of a supervised classification algorithms, a gene expression profile was identified strongly predictive for of a short interval to distant metastasis (‘poor prognosis’) in patients without tumour cells in local lymph nodes at diagnosis. This in vitro expression profile consists of genes involved in the processes of cell cycle, invasion, metastasis, and angiogenesis [Van't Veer et al. (2002) Nature 415, 530-536; Van de Vijver et al (2002) New Engl. J. Med. 347, 1999-2009].

Optical imaging is an extremely sensitive in vivo imaging tool for the assessment of tissue anatomy, physiology, and metabolic and molecular function. Fluorescent dyes can be detected at low concentrations while using low levels of radiation, generating a fluorescent signal which is harmless to the patient. In addition, optical instrumentation and novel contrast agents for optical in vivo imaging of diseases have likewise emerged on the market over the last years.

A wide variety of labels have been used for the optical imaging of organs and biological molecules. A recently developed class of compounds for in vivo optical imaging are Quantum dots. The use of quantum dots in biological imaging has been demonstrated in Goa et al. [(2004) Nature Biotechnology 22, 969-976] and is reviewed in e.g. Michalet et al. [(2005) Science 307, 538-544; Gao & Simmons (2005) Curr. Op. Biotech 16, 63-72; see also Chemy (2004) Phys. Med. Biol. 49, R13-R48].

Despite the availability of in vitro molecular biology techniques for the diagnosis and classification of diseases, there remains a further need for techniques which allow a detailed patient-specific diagnosis in vivo, i.e. based on a non-invasive whole-body analysis.

An object of the present invention is to provide alternative and/or improved methods and tools for improving diagnostic imaging in vivo.

An aspect of the present invention relates to tools, and methods for producing tools which allow accurate diagnostic imaging based on the simultaneous in vivo qualitative and/or quantitative detection of a plurality of biomedical markers such as the expression or non-expression of a plurality of genes (whether wild-type or mutated) or presence or absence of a plurality of carbohydrates or proteins or lipids (whether circulating or bound). Another aspect of the present invention relates to obtaining relevant parameters for an accurate diagnosis of a disease by a non-invasive imaging approach.

The methods of the present invention relate to quantitative and qualitative in vivo imaging, in order to determine the presence of a signature profile associated with a specific disease state, which has been determined based on molecular biological parameters of a tissue. This allows diagnosis, using a non-invasive diagnostic method, not only of a disease state, but more specifically of a subtype of a disease and a progression state. Providing such information which can be critical for the therapeutic approach to the disease and for outcome prognosis.

According to a particular embodiment of the invention, where the methods of the invention are applied to the diagnosis of cancer, they allow not only an identification of the presence of a cancer but also the classification as benign or malignant tumor, as well as, in the case of a malignant tumor, the determination of the differentiation grade and classification. Thus, using a non-invasive in vivo diagnostic method it is possible to define a suitable therapy and to predict the outcome parameters for treating a certain disease.

According to a first aspect of the invention a method is provided for the in vivo diagnosis of a disease or disorder based on a previously identified signature profile for said disease or disorder. Thus, the methods of the invention comprise a first aspect which is determining the signature profile for a disease state (for different types and progression stages of said disease or disorder) based on a plurality of factors which are biomedical targets or biomedical disease targets or biomedical disease diagnosis targets. Such an expression profile can for instance be a gene expression profile, such as an expression profile of a plurality of genes, e.g. expression or non-expression of the genes, or presence or absence of the gene products such as proteins (enzymes, receptors, structural proteins, etc) or an expression profile of carbohydrates, lipids, metabolites (whether bound or circulating). The second aspect involves determining whether said signature profile can be detected in a patient by in vivo imaging. This is achieved by making use of different biomedical targeting moieties such as gene and/or protein-specific and/or carbohydrate or lipid targeting moieties (whether bound or circulating and whether wild-type or mutated) each labelled with a compound emitting light at a different wavelength.

A specific embodiment of the method of the invention is the diagnosis of cancer, wherein the method of the invention allows the identification of a specific type of cancer (malignant, benign, primary, secondary, aggressive and non-aggressive tumor). Additionally, in the diagnosis of cancer, the methods of the invention allow the identification of the site of metastases homing.

According to another aspect, the invention provides methods for preparing kits for the in vivo diagnosis of a disease or disorder by expression profiling, whereby the kits comprise two or more, preferably a plurality of targeting moieties specifically directed against different factors. According to specific embodiments the method encompasses preparing or obtaining targeting moieties specific for factors that are selected from the group consisting of genes and/or proteins and/or carbohydrates and/or lipids and/or metabolites. These methods encompass a) determining the signature profile for different types and progression stages of said disease or disorder based on the target or factor profile, e.g. gene expression profile of a plurality of genes, presence or absence of a plurality of gene products such as proteins, and/or presence or absence of carbohydrates and/or lipids, and/or metabolites whether bound or circulating, b) providing targeting moieties which are specific for those targets or factors such as genes and/or proteins and/or carbohydrates and/or lipids, and/or metabolites making up said expression profile and c) labelling each targeting moiety with a compound emitting light at a different wavelength.

Depending on the diagnosis required, the signature profile associated with a specific disease, disease type or progression state is determined according to the present invention by identifying factors such as genes and/or proteins and/or carbohydrates and/or lipids that are differentially expressed between a healthy individual and a individual having said disease or disorder; and/or factors such as genes and/or proteins and/or carbohydrates and/or lipids that are differentially expressed in different stages of a disease, and/or factors such as genes and/or proteins and/or carbohydrates and/or lipids that are differentially expressed in diseases of which the biological basis is different but which lead to the same symptoms. The differential expression of the biomedical targets such as genes in each of these situations is qualitative and/or quantitative. According to a particular embodiment of the invention, the signature profile is determined in vitro, using techniques such as micro-array analysis and differential display methods.

Specific embodiments of the invention relate to signature profiles wherein the differentially expressed proteins are cell surface proteins, cell-surface receptors or secreted proteins.

Yet another aspect of the invention relates to kits for the in vivo diagnosis of a disorder by expression profiling, which kits comprise a plurality of targeting moieties directed against factors which are differentially expressed in health and disease whereby the different targeting moieties are differentially labelled. According to a particular embodiment the factors are selected from the group consisting of genes and/or proteins and/or carbohydrates and/or lipids and/or metabolites. According to a specific embodiment, each different targeting moiety is labelled with a compound emitting light at a different wavelength.

Specific embodiments of this aspect of the invention relate to kits wherein the compound(s) emitting light are selected from the group consisting of fluorescent dyes, quantum dots, and luminescent material, such as nanophosphor. A further specific embodiment of the invention relates to the use of quantum dots in the context of the present invention, as they allow the production of a large range of labels with different emission spectra which can be detected both qualitatively and quantitatively in a specific and sensitive manner. Specific quantum dots envisaged in the context of the present invention include those made of SeCd, CdS, HgTe and CdTe.

The targeting moieties used in the methods and kits of the present invention for the detection in vivo include proteins, antibodies or fragments or derivatives thereof, antisense molecules, aptamers, peptides or peptidomimetics, hormones and small molecules capable of binding a specific target. According to a specific embodiment of the invention, the targeting moiety is a monoclonal antibody or an antibody fragment or derivative such as a single chain Fv or a Fab fragment. Particularly useful for the in vivo detection methods of the present invention are humanized antibodies or antibody fragments.

According to a particular embodiment of the invention, the number of gene and/or proteins and/or carbohydrates and/or lipids and/or metabolite making up the signature profile (or representative selection thereof) to be detected in vivo is between 2 and 10, more particularly between 2 and 5, but signature profiles made up of between 5 and 10 or between 10 and 20 biomedical targets such as genes are also envisaged. The corresponding number of targeting moieties are present in the kits of the present invention.

A further aspect of the present invention relates to the use of the kits described herein in diagnostic imaging.

Yet a further aspect of the invention relates to the use of a plurality of specific targeting moieties directed against specific factors the expression of which is associated with a disease, wherein each different targeting moiety is labelled with a compound emitting light at a different wavelength, in the manufacture of a diagnostic kit for the in vivo diagnostic imaging of a tissue or an organ. More particularly the factors are genes and/or proteins and/or carbohydrates and/or lipids and/or metabolites.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

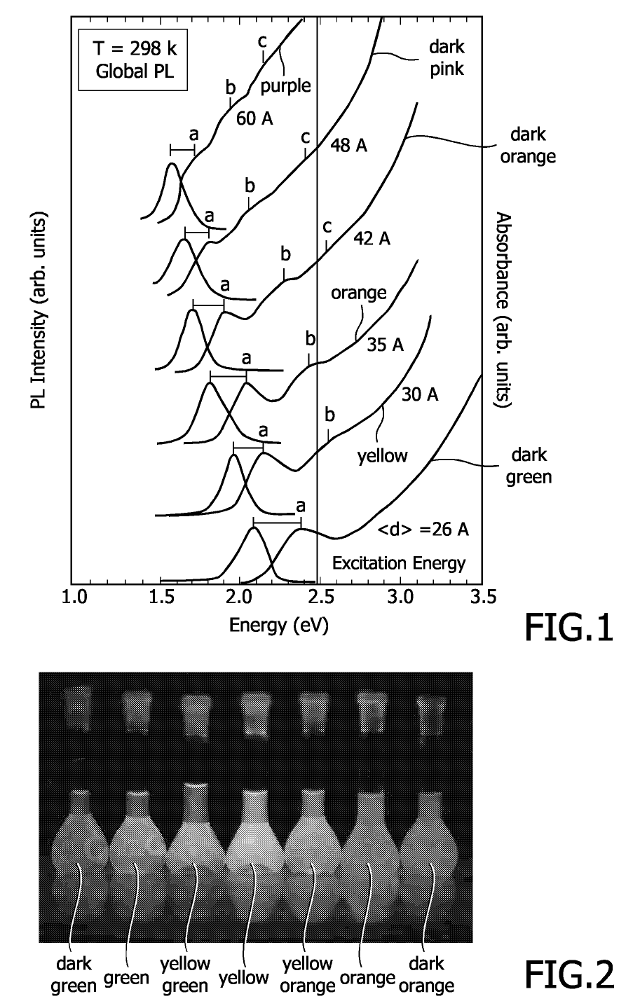

FIG. 1 The absorption and photoluminescence of different-sized QD samples.

FIG. 2 Qdots of different sizes excited with the same UV wavelength.

The present invention relates to the determination of signature profiles for different types and stages of progression of a disease or disorder and the use of this signature profile in in vivo diagnostic imaging.

A signature profile for a disease and/or the type and progression of a disease as used herein refers to an expression profile which is characteristic of the disease. Such a signature profile is the result of a qualitative and/or quantitative determination of the levels of expression of a number of individual factors subsequent comparison of these expression levels with an adequate control or reference (i.e. healthy individual, different type of the disease, different stage of the disease), thereby identifying which combination of factors allow the differentiation of the disease, type of disease or stage of disease over the control or reference. The factors referred to herein include any molecule which is differentially expressed in a disease state. According to a particular embodiment of the invention, the factors the expression of which makes up the signature profile are genes, proteins, carbohydrates, lipids, and/or metabolites. In the context of the present invention such a signature profile will involve the expression of at least two, more particularly between 2 and 10, for example 4, 5, 6 or 7, or optionally between 10 and 20 or between 20 and 30 factors.

The determination of the expression levels of a gene can be performed directly by measuring the protein content or indirectly by measuring the expression of gene at the DNA or mRNA level or by measuring the presence (qualitatively and or quantitatively) and/or the activity of the protein which is the gene product protein, again directly or indirectly (for example where the gene encodes an enzyme, the production of metabolites of an enzyme).

According to one embodiment of the present invention, the presence or absence of the signature profile associated with a particular disease or disorder, or a particular type or progression state thereof is identified in a patient by in vivo imaging using a plurality of specific targeting moieties capable of determining specific factors of the signature profile, whereby each different targeting moiety is labelled differentially. Differential labelling as used herein refers to the fact that for each factor or target a targeting moiety is used with a different label, which allows differentiation during the simultaneous detection of different factors. An example of differential labelling includes the use of compounds emitting light at different wavelengths. The binding of each of the targeting moieties to their target factor, which is detected optically, reflects qualitatively and/or quantitatively the presence of the different targets. Based hereon the presence or absence of the signature profiles in the patient in vivo can be determined.

According to the present invention a signature profile is generated for a specific condition, allowing very accurate identification of said condition using detection methods, such as in vivo detection methods, based thereon.

Because of the specificity of the signature profile of the present invention for the condition, the method of the present invention allows the diagnosis not only of a general disease condition (such as cancer, arthritis, infection with a foreign agent, etc.), but also allow the discrimination between different types of diseases, the different stages of the disease, and in some cases allow predictive diagnosis of the further evolution of the disease and/or allow the identification of susceptibility to a specific therapy.

The generation of the signature profile according to the present invention is performed by expression profiling and comparison of expression profiles of a specific disease or type of disease or progression state thereof with the adequate control or reference. Thus, depending on the type of signature profile required, the signature profile can be obtained by identifying factors such as genes, proteins, carbohydrates, lipids and/or metabolites that are differentially expressed in a tissue when compared between a healthy individual and an individual having a specific disease or disorder, by identifying factors that are differentially expressed in a tissue in different stages of a disease or by identifying factors that are differentially expressed in one or more tissues in diseases of which the biological basis is different but which cause the same symptoms in the patient. In each of these situations, the differential expression can be the result of differences in either qualitative or quantitative expression of a factor or both.

The expression profile of tissue in a certain condition can be obtained either in vitro or in vivo. According to a particular embodiment of the present invention, the expression profile of a tissue is obtained in vitro. An expression profile for a specific condition can be determined in vitro for instance by comparing biological material from an affected tissue with that of a control tissue using methods such as but not limited to micro-array techniques, differential display methods and proteomic techniques, such as the comparison of two dimensional protein gels patterns combined with mass spectrometry. Such comparisons are preferably based on multiple samples, in order to improve the reliability of the observed differences; the data obtained by these methods is then optionally analyzed by suitable data analysis systems (e.g. using algorithms such as learning algorithms). These methods allow the qualitative and quantitative determination and differentiation of the expression of a large number of factors simultaneously. In vitro gene expression profiles have been identified for specific tissues for different diseases in the art, such as for instance for different types of cancer [Van't Veer et al. (2002) Nature 415, 530-536].

According to a particular embodiment, the methods of the present invention include the application and transfer of information obtained in vitro to an in vivo detection setting. The use of an in vivo detection method is envisaged which allows the (quantitative and qualitative) detection of the signature profile. The invention thus provides tools and methods to obtain classification and outcome parameters for a specific disease (such as, but not limited to cancer) by non-invasive imaging approaches, thereby avoiding the surgical intervention to withdraw a tissue section of the patient's body.

The expression profiling and subsequent comparison with the reference allows the identification of a particular set or combination of factors for which a certain (qualitative and/or quantitative) expression profile can be linked to a specific condition. According to the present invention a selection is optionally made within this set of factors to identify those factors the expression of which allow for detection in vivo, i.e. by targeting with a targeting moiety. Such a targeting can be of a gene, of the corresponding mRNA, of the corresponding gene product, of the glycosylation of said gene product or of a substrate or metabolite of said gene product. In some situations the expression of a gene can also be detected by targeting a compound directly associated with the expression of the gene (such as the metabolite of an enzyme). The following selection criteria are relevant for the selection of suitable factors or targets to be analyzed in the in vivo diagnostic method of the present invention: the localization of the factor in or outside the cell, the effect of the binding of a targeting moiety to the factor in vivo (i.e. on the metabolism or functioning of a cell), and this both for affected and non-affected cells or tissues; other practical considerations can also determine the choice of factors or targets selected, such as e.g. the availability and size of a targeting moiety specifically binding to the factor.

The types of molecules envisaged to be detected as factors in the in vivo method of the present invention include cell surface proteins, receptors, secreted proteins, cytosolic proteins, nuclear proteins, carbohydrates, metabolites, etc. According to a specific embodiment of the invention, the factors or targets are secreted proteins (growth factors and cell signalling molecules) and/or cell surface proteins such as membrane receptors, cell adhesion molecules, and other cell surface polypeptides, which allow easy access for targeting moieties. Alternatively, however, the factor is an internal molecule such as a component of a signalling cascade such as a kinase, phosphatase or a transcription factor.

According to the present invention the selected combination of factors the quantitative and/or qualitative expression of which allows the specific identification of a certain condition. However, this does not exclude, within the combination of factors to be detected in the in vivo method of the present invention, the presence of factors within a signature profile which serve as a reference e.g. by allowing the identification of a certain cell or tissue or of a certain metabolic reaction. Thus, according to a particular embodiment, the plurality of factors or targets selected for in vivo detection based on the signature profile, additionally comprises a target which has a constant expression both in healthy and disease conditions, or in different stages of he disease. According to a specific embodiment this target is specific for the organ or cell type under investigation.

An example of a factor is GFAP (Glial fibrillary acidic protein), which is specifically expressed on the cellular surface of astrocytes. This protein can be used differentiate between neurons and astrocytes.

Different targeting moieties are envisaged for use in the context of the present invention. Targeting moieties which are suitable for the detection of the expression of genes at the DNA or mRNA level are typically antisense molecules. Oligonucleotides can be labelled with quantum dots through the strong specific interaction of streptavidin and biotin. Alternatively, DNA is coupled to microspheres comprising quantum dots.

Targeting moieties which are suitable for the detection of the expression of proteins or carbohydrates are for example antibodies (and antibody-fragments), peptides, hormones, receptor-ligands, aptamers and small molecules such as enzyme inhibitors, receptor agonists and receptor antagonists. According to a certain embodiment, the targeting moieties bind with cell surface proteins, such as receptors and membrane proteins, in particular proteins involved in cell-cell interactions. In a preferred embodiment the targeting moieties are low molecular weight proteinaceous compounds such as antibody fragments, ligands, and peptides representing the binding part of a ligand. In order to detect intracellular targets using in vivo imaging techniques, the targeting moiety has to enter the cell. The same compounds and methods as described above are suitable for this purpose.

Moreover, it is envisaged that for some conditions, intracellular targets are of use for the detection of lysed cells. For instance, the development of cancers is hallmarked by a high rate of dividing cells and consequently a high number of lysed cells. Using one or more targeting moieties which are not internalized by the non-lysed cell and but which bind to intracellular proteins allows the discrimination between organs wherein a high proliferation and a low proliferation rate occurs and which is indicative for the aggressive character of a tumor.

By the high-affinity binding of the targeting moiety to its molecular target, a highly site-specific signal in the imaging process is achieved.

According to the present invention, the specific (quantitative and or qualitative) expression of each of a combination of factors is detected simultaneously in vivo, more particularly by in vivo imaging. Thus, the methods and compounds of the present invention rely on the use of labels and corresponding detection methods that allow simultaneous differential detection of different factors. Optimally such labels and detection methods thereof allow both qualitative and quantitative detection of a number of different factors. According to a particular embodiment of the invention this is achieved by optical imaging and the use of light-emitting labels. Optical imaging is an extremely sensitive in vivo imaging tool for the assessment of tissue anatomy, physiology, and metabolic and molecular function. Optical imaging on living beings is generally based on light emission within the UV (ultraviolet) and the NIR (near-infrared) spectral region. The penetration depth of light into living body tissue depends on the wavelength applied due to the fact that scattering and absorption incidences are functionally correlated to the used wavelength. In the spectral range less than 600 nm, light absorption in body tissue is relatively high resulting in a small penetration depth of hundreds of micrometers up to a few millimetres, which is only suitable for the superficial investigation of tissue or organ surfaces. To image larger tissue volumes, light within the NIR spectral range (700-900 nm) is more appropriate and gives a higher penetration depth into the assessed material reaching up to a few centimetres. Thus, the identification of changes of morphology and/or function of a tissue in a living organism is possible.

According to the present invention, the targeting moieties are each linked to a contrast enhancing material (label), allowing the simultaneous qualitative and or quantitative detection of each of the factors or targets. According to a specific embodiment of the present invention it is envisaged that all targeting moieties used for the detection of the combination of factors to identify in vivo the presence of a signature profile are labelled with a label of the same type (that is detectable using the same imaging modality. In order to determine expression of factors in a qualitative and quantitative manner using optical imaging, different targeting moieties are each labelled with optical labels emitting light at different wavelengths. Suitable labels for optical imaging in accordance with the present invention are for example fluorescent dyes and quantum dots.

According to one embodiment the labels for optical imaging are fluorescent labels. Fluorescent molecules which emit light at different wavelength and which are suitable to label different targeting moieties are known in the art and available commercially (e.g. from Sigma-Aldrich).

According to another embodiment, the labels for optical imaging to be used in accordance with the present invention are quantum dots, also referred to as Qdots or QDs—(These are commercially available e.g. Evident Technologies). These are crystalline semiconductor clusters typically derived in the II/VI and III/V material systems. The most commonly used semiconductors are CdSe, CdS, HgTe, CdTe, InP and InAs. The structural size of the quantum dots needs to be in the order of the exciton Bohr radius in the respective material, which typically amounts to sizes from 1-10 nm, in order to obtain quantization effects. Using appropriate synthesis procedures well-crystallized particles with quasi-spherical geometry can be produced [Murray et al. (1993) J. Am. Chem. Soc. 115, 8706; Micic et al. (1996) Appl. Phys. Lett. 68, 3150; Vossmeyer et al. J. Phys. Chem. 98, 7665 (1994)]. Although the crystallization process can be well controlled, the synthesis typically leads to a distribution of sizes. This distribution leads to a broadening of the electronic states and thus to the optical response of a QD ensemble. This size distribution can be narrowed by means of size selective precipitation to about 20-35 nm FWHM (full widths at half maximum). Qdots can be synthesized in so called “core-shell”-Systems where the particles are surrounded with a shell material that has a higher bandgap, e.g. ZnS. In this systems the optical properties of the particles are defined by the choice of material and size of the core.

The shell modifies the surface of the core in such a way that energetically low lying surface trap states, that are independent of the size and close to the bulk bandgap of the semiconductor material, are decreased. This results in higher fluorescence emission efficiencies and chemical and photo-stability of the Qdots. In addition, further coatings of organic or polymeric materials can be introduced that e.g. increase the colloidal stability of particles in solution by preventing agglomeration, provide an effective electronic barrier (helping to confine the electron and hole wavefunctions to the core of the nanocrystal, by passivating surface trap states), tune their solubility in different media, offer linker chemistry for conjugation of biomolecules, and reduce unspecific binding. It has been shown that biomolecules bound to Qdots like antibodies keep their biological activity and that they can be used in common assays with slight modifications of the protocols. Highly efficient Qdots can be prepared with identical surface properties and linker chemistry independent of their emission color.

The above-mentioned quantization effect results from a dependence of the bandgap of colloidal Qdots and therefore their emission wavelength depends on the particle size. With decreasing particle size the emission energy increases. Therefore, using only a few different semiconductor materials practically every emission wavelength from UV to IR can be realized. The change in absorption and emission for a series of InP QD ensembles with different main sizes is reproduced in FIG. 1. As can be seen a blue-shift of both the emission maximum as well as the absorption onset is observed, consistent with the predictions above mentioned. This manifests the size dependence of the optical bandgap.

In addition to their small and symmetric emission band, their very high photostability and extinction coefficient as well as their very high quantum yield, Qdots absorb in principal every wavelength shorter than their emission wavelength. Therefore, all Qdot particles that emit in the visible and IR spectral region can be exited simultaneously with the same UV energy (see FIG. 2). Especially this property simplifies the application of Qdots in multiplexed assays and reduces the cost of equipment a lot, because there is no longer a need for different excitation sources (lasers) for different emission colors. Therefore, the use of Qdots has a number of advantages and enables multiplexing approaches.

Quantum dots can be applied in a biological setting both in vitro and in vivo. The rapid labelling of whole cell populations with specific colors can be performed using peptide translocation domains or cationic lipids which are efficient facilitators of endocytosis. A better specificity and efficiency has been obtained using functionalized qdots.

Another strategy consists of cross-linking primary antibodies to qdots. This can be performed in two different ways The first approach involves the biotinylation of the primary antibody directed against the target, which is subsequently attached to avidin-coated qdots. The second approach involves engineering an adaptor protein with both binding affinity to the Fc region of antibodies and electrostatic interactions with charged qdots. To reduce the size of qdot probes, ligands of surface receptors are bound to qdots via a biotinstreptavidin link or by direct crosslinking as described in the art. For instance, EGF-labeled qdots can be used to study receptor-mediated signal transduction in different cancer cell lines. Some proteins can be recognized by peptides, and these can be used peptides for qdot functionalization as described in the art. In the absence of such peptide sequences or of an identified ligand, the target molecule can be engineered to include a recognizable polypeptide, for example by fusion of an avidin polypeptide chain to the glycosylphosphatidylinositol (GPI)-anchoring sequence of human CD14 receptors [Pinaud et al. (2004) J. Am. Chem. Soc. 126, 6115]. Also, biotinylated peptide-coated qdots can be used to label the avidin receptors expressed in the cytoplasmic membrane of cells. The same concepts can be used to target and label cytoplasmic or nuclear targets. However, if internalization is desired qdots need to (i) enter the cell cytoplasm and (ii) reach their target without being trapped in the endocytic pathway (Jaiswal et al. cited above). Peptide-qdots can be used to target tissue-specific vascular markers (lung blood vessels and cancer cells) by intravenous injection in live mice as described in the art. Qdots emit light in the visible spectrum and a spectral demixing algorithm can be used to separate tissue autofluorescence from qdot signal in organs as described in the art. This problem can be circumvented using NIRemitting qdots (850 nm) [Kim et al. (2004) Nature Biotechnol. 22, 93-97]. Qdots can be also be used in in vivo cross-linking strategies developed for dyes -such as the use of biarsenical ligands targeted against tetracysteine motifs [Adams et al. (2002) J. Am. Chem. Soc. 124, 6063-6076 (2002)], Ni2+-nitrilotriacetic acid moieties targeted against hexahistidine motifs [Kapanidis et al. (2001) J. Am. Chem. Soc. 123, 12123-12125]. The development of affinity pairs orthogonal to the biotin-avidin pair, in which one component could be easily attached or fused to the target protein, is also applicable in the present invention. Several examples of such pairs developed by molecular evolution have been reported, with fusion peptides ranging from about 200 amino acids [single chain fragment antibody targeted against fluorescein, dissociation constant Kd of about 50 fM [Boder et al. (2000) Proc. Natl. Acad. Sci. U.S.A. 97, 10701-10705] down to about 30 amino acids [peptide hairpin against Texas Red, Kd of about 25 pM [Marks (2004) Chem. Biol. 11, 347-356].

Alternative imaging modalities for use in accordance with the present invention include, nuclear imaging, MRI and CT. Suitable labels for Nuclear Imaging are for example ⁹⁹Technetium, ¹²⁵Iodine (SPECT), ¹⁸Fluor and ¹¹Carbon (PET) Suitable labels are for ultrasound Imaging are gas filled microbubbles and liposomes. Labels for MRI are typically Gd-complexes, iron oxide (magnetic) and nanoparticles. Labels for CT are typically iodinated compounds and lipids.

As indicated above, according to a specific embodiment of the present invention it is envisaged that all targeting moieties used for the detection of the combination of targets to identify in vivo the presence of a signature profile are labelled with a label of the same type (that is detectable using the same detection method).

According to an alternative embodiment of the invention however, one or more targeting moieties, more particularly those used to identify a non-disease associated target, can also comprise another type of label. For example in a setting wherein quantum dots are used as labels for the genes of the signature profile, the moiety which binds to a protein with a constant expression level is labelled for instance with microbubbles or with compounds for MRI. This allows the visualization of the organ and can also be used as an in internal standard for concentration determinations.

Alternatively, according to the present invention, the detection of the expression of the genes making up the signature profile can also be performed using a plurality of targeting moieties which are labelled with labels for different imaging modalities. For example, one targeting moieties is labelled with a fluorescent dye, a second targeting moieties is labelled with a radioactive isotopes and a third targeting moiety is labelled with ultrasound microbubbles. The binding of the different targeting moieties to their targets is then determined quasi-simultaneously using a combination of optical imaging, radiography and ultrasound.

One aspect of the invention relates to a method for the specific in vivo diagnosis of a disease or disorder, based on determining in vivo the expression profile of a set of factors, based on a previously identified signature profile for said disease or disorder. The in vivo detection is achieved, for instance, by using a plurality of targeting moieties specific for these factors each labelled with a compound emitting light at a different wavelength.

The method of the invention allows the identification of a very specific disease condition. For instance the signature profile can be obtained so as to allow the identification of the disease type, to provide information on the disease progression, outcome and/or appropriate treatment. An important application envisaged for the method of the present invention is in the diagnosis of cancer.

Indeed, as evidenced by the in vitro expression profiles identified in the art, the methods of the present invention allow the discrimination between malign and benign tumours and the identification of the grade of a certain tumor. The expression of specific factors has also been demonstrated to be linked to the potential of a tumor to undergo metastasis. The expression level of a few genes and/or proteins (e.g., interleukin-11 (IL-11), connective tissue growth factor (CTGF), chemokine receptor-4 (CXCR-4), matrix metalloproteinase-1 (MMP-1), or the von Hippel-Lindau tumour suppressor protein (pVHL) is indicative of a highly increased incidence for a tumour to establish distant metastasis [Van't Veer (2003) Nature Med. 9, 999-1000; Bernards (2003) Nature 425, 247-248]. The expression of certain genes has also been shown to indicate the preferred location of the metastasis site of a certain tumor within the body [Kang et al. (2003) Cancer Cell 3, 537-549.]. Finally, a more specific cancer diagnosis allows tailor-made therapeutic regimes: chemotherapy reduces the risk of distant metastases by approximately one-third; however, 70-80% of patients receiving this treatment would have survived without it. By the use of in vitro gene expression analysis in primary breast tumours and the application of supervised classification algorithms, a gene expression signature was identified strongly predictive for of a short interval to distant metastasis (‘poor prognosis’) in patients without tumour cells in local lymph nodes at diagnosis. This in vitro expression signature consists of genes involved in the processes of cell cycle, invasion, metastasis, and angiogenesis [[Van't Veer et al. (2002) Nature 415, 530-536; Van de Vijver et al (2002) New Engl. J. Med. 347, 1999-2009]]. The reported findings provide a strategy to select patients who could benefit from therapy. The methods of the present invention moreover make it possible to identify expression signature in an individual patient with a non-invasive in vivo detection method.

The in vivo expression diagnostic tools of the present invention are used as an alternative or in combination with one or more of the following diagnostic techniques: physical examination, biochemical and histopathological investigations, and diagnostic imaging techniques routinely diagnosing cancer tissue based on its morphology.

### EXAMPLE 1

### Quantum Dot-Labelled Targeting Moieties for In Vivo Expression Profiling of Breast Cancer

The set of 70 genes which have been described for in vitro expression profiling of breast cancer (Van't Veer et al. Nature 415, 530-536) were analyzed for their suitability to be used in in vivo expression profiling:

The target genes depicted in Table 1 are secreted or extracellular proteins selected from the above-mentioned set of 70 genes.

The availability of targeting moieties is further determined for these targets and each of these targeting moieties is then labelled to a different quantum dot.

TABLE 1
targets from breast cancer imaging

| | | | |
|---|---|---|---|
| | Targeting | | |
| Target | moiety | label | Emission |

Genes upregulated in sporadic breast tumors with distant metastatis

| | | | |
|---|---|---|---|
| Flt 1 | VEGF | QD-Color: Gallium | ~850 nm |
| | | NIR | |
| MMP9 | antibody | QD-Color: Snake | ~950 nm |
| | | Eyes NIR | |

enes upregulated in sporadic breast tumors without distant metastatis

| | | | |
|---|---|---|---|
| FGF-18 | antibody | QD-Color: Maple | ~620 nm |
| | | Red-Orange) | |
| IGFB5 | antibody | QD-Color: Cortland | ~640 nm |
| | | Red | |
| ESM1 | antibody | QD-Color: Empire | ~660 nm |
| | | Red | |
| CFFM4 | antibody | QD-Color: Adam's | ~720 nm |
| Membrane-Spanning | | Apple Red | |
| 4-Domains, | | | |
| Subfamily A, | | | |
| Member 7 | | | |
| TGFbeta 3 | antibody | QD-Color: Gallium | ~850 nm |
| | | NIR | |
| WNT1-Inducible | antibody | QD-Color: Snake | ~950 nm |
| Signaling Pathway | | Eyes NIR | |
| Protein 1 | | | |

A set of 2-10 of these markers is then used to detect in vivo the signature profile of the different types of cancer in patients having been diagnosed with cancer, so as to identify the optimal therapeutic regime for each patient.

1. A method for the in vivo diagnosis of a disease or disorder, comprising the steps of:
determining the signature profile for different types and progression stages of said disease or disorder based on the expression profile of a plurality of factors in comparison to a control, with the signature profile indicating which combination of factors allows the differentiation of the disease or disorder, of the type of the disease or disorder or of the stage of the disease or disorder over the control
determining whether said signature profile can be detected in a patient, making use of different targeting moieties specific for each of said factors, each of said targeting moieties being differentially labelled.

2. The method according to claim 1, wherein said factor is selected from the group consisting of genes, proteins, carbohydrates, lipids, and metabolites.

3. The method according to claim 1 wherein each of said specific targeting moiety is labelled with a compound emitting light at a different wavelength, or a compound labelled with different radioactive isotopes (for the purpose of PET, or SPECT Imaging), or a compound labelled with groups with different magnetic properties (for the purpose of MR Imaging).

4. The method according to claim 1 wherein the disease or disorder is cancer.

5. The method of claim 3, wherein said method results in the identification of a type of cancer selected from the group of consisting of a malignant, benign, primary, secondary, aggressive and non-aggressive tumor.

6. The method according to claim 3, wherein a signature profile identifies the site of metastases homing.

7. A method for preparing a kit for the in vivo diagnosis of a disease or disorder by expression profiling, said kit comprising a plurality of targeting moieties each specific for a factor, said method comprising the steps of:
a) determining the signature profile for different types and progression stages of said disease or disorder based on the expression profile of a plurality of factors in comparison to a control with the signature profile indicating which combination of factors allows the differentiation of the disease or disorder, of the type of the disease or disorder or of the stage of the disease or disorder over the control;
b) providing a targeting moiety which is specific for each of said factors.
c) labelling each of said targeting moiety differentially

8. The method according to claim 7, wherein said factors are selected from the group consisting of genes, proteins, carbohydrates, lipids, and metabolites.

9. The method according to claim 7, wherein said differential labelling comprises labelling each of said specific targeting moiety with a) a compound emitting light at a different wavelength, b) a compound labelled with different radioactive isotopes (for the purpose of PET, or SPECT Imaging), or c) a compound labelled with groups with different magnetic properties (for the purpose of MR Imaging).

10. The method of claim 7 wherein said signature profile is determined by identifying:
1) factors that are differentially expressed between a healthy individual and a individual having said disease or disorder; and/or
2) factors that are differentially expressed in different stages of a disease,
3) factors that are differentially expressed in diseases of which the biological basis is different but which lead to the same symptoms, whereby said differential expression is qualitative and/or quantitative.

11. The method of claim 7, wherein said signature profile is determined in vitro.

12. The method of claim 11, wherein said signature profile is determined by micro-array analysis.

13. The method of claim 7, wherein said factors are cell surface proteins, cell-surface receptors or secreted proteins.

14. A kit for the in vivo diagnosis of a disease or a disorder by expression profiling, said kit comprising a plurality of targeting moieties each specific for a different factor of a signature profile for different types and progressions of a disease or disorder, with the signature profile indicating which combination of factors allows the differentiation of the disease or disorder, of the type of the disease or disorder or of the stage of the disease or disorder over a control, characterized in that each of said targeting moieties is labelled with a compound emitting light at a different wavelength.

15. The kit according to claim 14 wherein said compound emitting light is selected from the group consisting of fluorescent dyes, quantum dots, luminescent material, radionuclides or isotopes, paramagnetic and/or super-magnetic materials.

16. The kit according to claim 15, wherein said luminescent material is a nanophosphor.

17. The kit according to claim 15 wherein said quantum dots are selected from the group consisting of SeCd, CdS, HgTe and CdTe.

18. The kit according to claim 14, wherein the targeted contrast agent is labelled with different contrast enhancing materials.

19. The kit according to claim 14, wherein the targeting moiety is selected from the group consisting of a protein, an antibody or a fragment or derivative of an antibody, an antisense molecule, an aptamer, a peptide or peptidomimetic, a hormone and a small molecule capable of binding specifically to a target.

20. The kit according to claim 19, wherein said antibody is a monoclonal antibody.

21. The kit according to claim 19, wherein said antibody fragment or derivative thereof is a single chain Fv or an Fab fragment.

22. The kit according to claim 19, wherein the antibody, antibody fragment or derivative thereof is human or humanized.

23. The kit according to claim 14 wherein the number of gene and/or protein specific targeting moieties is between 2 and 5.

24. The kit according to claim 14 wherein the number of gene and/or protein specific targeting moieties is between 2 and 10.

25. Use of the kit of claim 14 for in vivo diagnostic imaging.

26. Use of a plurality of gene and/or protein specific targeting moieties, wherein each different targeting moiety is labelled with a compound emitting light at a different wavelength and wherein each moiety recognizes a factor of a signature profile, with the signature profile indicating which combination of factors allows the differentiation of the disease or disorder, of the type of the disease or disorder or of the stage of the disease or disorder over the control, in the manufacture of a diagnostic kit for the in vivo diagnostic imaging of a tissue or an organ.
